SEQUENCE LISTING (3 sequences)

SEQ ID NO: 1 - protein, 418 aa, Homo sapiens
MQALVLLLCIGALLGHSSCQNPASPPEEGSPDPDSTGALVEEEDPFFKVPVNKLAAAVSNFGYDLYRVRSSMSPTTNVLLSPLSVATALSALSLGAEQRTESIIHRALYYDLISSPDIHGTYKELLDTVTAPQKNLKSASRIVFEKKLRIKSSFVAPLEKSYGTRPRVLTGNPRLDLQEINNWVQAQMKGKLARSTKEIPDELSLLLLGVAHFKGQWVTKFDSRKTSLEDFYLDEERTVRVPMMSDPKAVLRYGLDSDLSCKIAQLPLTGSMSLIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 2 - protein, 44 aa, Homo sapiens
VLLSPLSVATALSALSLGAEQRTESIIHRALYYDLISSPDIHGT

SEQ ID NO: 3 - protein, 44 aa, Homo sapiens
VLLSPLSVATALSALSLGAEQRTESILHRALYYDLISSPDIHGT

(19) United States
(12) Patent Application Publication
(10) Pub. No.: US 2008/0293626 A1
(43) Pub. Date: Nov. 27, 2008

(54) Pigment epithelium-derived factor as a therapeutic agent for vascular leakage

(75) Inventors: Patrick Tong, Baltimore, MD (US); Hua Liu, Baltimore, MD (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 11/413,118

(22) Filed: Apr. 26, 2006

Related U.S. Application Data
(63) Continuation of application No. PCT/US04/36245, filed on Oct. 29, 2004.
(60) Provisional application No. 60/515,374, filed on Oct. 29, 2003.

(51) Int. Cl. A61K 38/00; A61P 3/10

(52) U.S. Cl. 514/12

(57) ABSTRACT

The present invention relates to method of treating a patient with a condition involving increased vascular permeability or increased angiogenesis comprising administering to the patient a therapeutically effective amount of PEDF, PEDF 44 AA peptide, a homolog of the PEDF 44 AA peptide, a homolog of the PEDF 44 AA peptide wherein amino acid residues glutamate the (101) amino acid position, isoleucine at the (103) amino acid position, leucine at the (112) and serine at the (115) amino acid position are unchanged, or an agent that activates the PEDF receptor. Conditions for treatment include, but are not limited to, sepsis acute respiratory distress syndrome, nephrotic syndrome, diabetic neuropathy, preproliferative diabetic retinopathy, cancer or proliferative diabetic retinopathy.

### PRIORITY

The present application claims priority to U.S. Provisional Application No. 60/515,374, filed Oct. 29, 2003.

### FIELD OF THE INVENTION

The field of the invention relates to compositions and methods that are useful in the treatment or prevention of conditions involving vascular permeability, angiogenesis and/or neuropathic disorders.

### BACKGROUND OF THE INVENTION

Vascular permeability and its regulatory control are central to homeostasis. Increases in vascular permeability play a key role in the development of sepsis-associated hypotension, acute respiratory distress syndrome, nephrotic syndrome, diabetic nephropathy, and diabetic retinopathy. Although the physiologic importance of maintaining the normal vascular integrity is well-appreciated, an understanding of how vascular integrity is maintained, and whether vascular permeability can be down regulated, remains elusive.

The activity of vascular endothelial growth factor (VEGF) in promoting vascular permeability is well established¹. In addition to promoting vascular permeability in guinea pig skin¹, VEGF is an important mediator of angiogenesis in vivo^{2,3}, and has neurotrophic/neuroprotective activity⁴⁻⁶. VEGF exerts its effects on endothelial cells via two tyrosine kinase receptors, the fins-like tyrosine kinase-1 (Flt-1; VEGFR-1) and fetal liver kinase-1 (Flk-1/KDR; VEGFR-2)⁷. VEGFR-2 is the dominant signaling receptor for many of VEGF's biological activities, including vascular permeability^{8,9}.

Pigment epithelium-derived factor PEDF), a 418-amino acid 50-kDa glycoprotein, is a member of the serine protease inhibitor (serpin) family^{10,11}. Although PEDF has a putative protease-sensitive loop, unlike classical serpins such as α1-antichymotrypsin (ACT), PEDF lacks protease inhibitory activity. Among serpins, this absence of anti-protease activity is not unique to PEDF; heat shock protein 47 (HSP47), a collagen-specific chaperone protein from the serpin family, also lacks anti-protease activity¹². PEDF was originally identified as an extracellular component of the retinal interphotoreceptor matrix^{13,14} PEDF functions in promoting neurite outgrowth in Y79 retinoblastoma cells^{15,16} More recently, PEDF has been found to be a potent anti-angiogenic factor¹⁷, effectively inhibiting neovascularization in a murine model of ischemia-induced retinopathy¹⁸.

The biological activities of VEGF and PEDF are similar in some cases, but antagonistic in other cases. Both VEGF and PEDF are active in angiogenesis and motoneuron survival. In the vascular endothelial cell system, VEGF and PEDF have counterbalancing proangiogenic and anti-angiogenic activities, respectively^{17,19-23}. In motoneurons, both PEDF and VEGF function in concert as neurotrophic/neuroprotective agents²⁴⁻²⁷. Although the relationships between PEDF and VEGF in angiogenesis and motoneuron survival have been established, it is unknown what effect PEDF has on VEGF's activity in vascular permeability.

Given the prevalence of vascular permeability and angiogenesis related disorders, there remains a need for an effective prophylactic and therapeutic treatment of these disorders, in particular those related disorders associated with both vascular permeability and neovascular complications, such as preproliferative and proliferative diabetic retinopathy.

### SUMMARY OF THE INVENTION

Vascular permeability plays a key role in a wide array of life-threatening and sight-threatening diseases. Vascular endothelial growth factor (VEGF) can increase vascular permeability. The discovery underlying the present invention relates to the finding that pigment epithelium-derived factor (PEDF) effectively abated VEGF-induced vascular permeability. In particular, a 44-amino acid region of PEDF confers both the anti-vasopermeability and the anti-angiogenic activities. Additionally, 4 amino acids (glutamate₁₀₁, isoleucine₁₀₃, leucine₁₁₂ and serine₁₁₅) were identified as critical for both activities. PEDF, or a derivative, could potentially abate or restore vision loss from diabetic macular edema, and the neovascular form of age-related macular degeneration. Furthermore, PEDF and/or a 44 amino acid (AA) peptide thereof represents a new therapeutic approach to sepsis associated hypotension, nephrotic syndrome, and other sight-threatening and life-threatening diseases resulting from excessive vascular permeability and/or angiogenesis.

The present invention relates to method of treating a patient with a condition involving increased vascular permeability comprising administering to the patient a therapeutically effective amount of PEDF, PEDF 44 AA peptide, a homolog of the PEDF 44 AA peptide, a homolog of the PEDF 44 AA peptide wherein amino acid residues glutamate at the 101 amino acid position, isoleucine at the 103 amino acid position, leucine at the 112 and serine at the 115 amino acid position are unchanged, or an agent that activates the PEDF receptor. Conditions for treatment include, but are not limited to, sepsis, acute respiratory distress syndrome, nephrotic syndrome, diabetic neuropathy, preproliferative diabetic retinopathy, and the neovascular form of age-related macular degeneration.

The present invention also relates to method of treating a patient with a condition involving increased angiogenesis comprising administering to the patient a therapeutically effective amount of PEDF 44 AA peptide, a homolog of the PEDF 44 AA peptide, a homolog of the PEDF 44 AA peptide wherein amino acid residues glutamate at the 101 amino acid position, isoleucine at the 103 amino acid position, leucine at the 112 and serine at the 115 amino acid position are unchanged, or an agent that activates the PEDF receptor. Conditions for treatment include, but are not limited to, cancer and proliferative diabetic retinopathy.

Further, the present invention relates to screening assays for the identification of candidate agents that can interact and activate the PEDF receptor. These candidate agents can include any molecule, protein or pharmaceutical (i.e., small molecule chemical) with the capability of mimicking or effectuating the biological action of PEDF.

Other and further aspects, features and advantages of the present teachings will be apparent from the following description of the various embodiments of the present teachings given for the purpose of disclosure.

### BRIEF DESCRIPTION OF THE FIGURES

FIG. 1 illustrates that PEDF qualitatively inhibits VEGF-induced retinal vascular permeability, wherein recombinant mouse VEGF₁₆₄ (VEGF) was injected into one eye, and the test reagents coinjected into the contralateral eye; fluorescein angiography revealed the degree of leakage into the retina and vitreous, whereas VEGF induced vascular leakage to a much higher degree than that observed with PBS (a) other reagents were co-injected with VEGF: recombinant human PEDF (PEDF) (b); α1-antichymotrypsin (ACT) (c); and heat shock protein 47 (HSP47) (a); all photographs are characteristic of the results of 4 or more mice.

FIG. 2 illustrates that PEDF quantitatively inhibits VEGF-induced retinal vascular permeability, wherein twenty-four hours after intravitreous injection of recombinant mouse VEGF₁₆₄ (VEGF) into one eye and test reagents into the contralateral eye, the amount of retinal Evans blue characterizes vascular leakage, the amount of VEGF-induced vascular leakage above control CBS) was set to 100%; vascular leakage with PBS injection was set to 0% (n=29); a second reagent was co-injected with VEGF to test its effect on vascular permeability; human PEDF (n=26), but not ACT (n=27) nor HSP47 (n=28), obliterated the VEGF-induced vascular permeability; data are means ±SE, with n representing the number of mice in each group; *, P<0.05 compared with vascular permeability induced by VEGF.

FIG. 3 illustrates that a 44-amino acid peptide from human PEDF, PEDFₚₑₚ, effectively inhibits VEGF-induced retinal vascular permeability wherein (a) PEDFₚₑₚ co-injection effectively inhibits VEGF-induced fluorescein leakage from the retinal vasculature (upper panels); mouse eye injected with both VEGF and ACTₚₑₚ, a peptide from ACT in the corresponding region of PEDFₚₑₚ, showed no discernible difference from the eye injected with VEGF alone (lower panels); (b) PEDFₚₑₚ co-injection effectively inhibits VEGF-induced vascular permeability quantitatively by Evans blue assay; the VEGF-induced increase in Evans blue was effectively inhibited with co-injection of PEDFₚₑₚ (n=26) with VEGF; no inhibition of VEGF-induced vascular permeability was observed with ACTₚₑₚ co-injection (n=28), in equimolar amounts as PEDFₚₑₚ; data are means ±SE, with n representing the number of mice in each group; *, P<0.05 compared with vascular permeability induced by VEGF.

FIG. 4 illustrates that substitution of 4 amino acid residues on PEDFₚₑₚ with corresponding residues from ACT or HSP47, abolishes modulation of vascular permeability, wherein four amino acid residues were substituted in PEDFₚₑₚ to give CHIMERAₚₑₚ; corresponding sequences of PEDFₚₑₚ, CHIMERAₚₑₚ, ACTₚₑₚ, and HSP47 are aligned (a); identical and similar amino acid residues are shaded in dark and light blue, respectively and amino acid substitutions in PEDFₚₑₚ substituted to give CHIMERAₚₑₚ are highlighted in yellow; the crystallographic structures of PEDF (Protein Data Bank ID 1IMV) and ACT (Protein Data Bank ID 1QMN) are shown (b); PEDFₚₑₚ and ACTₚₑₚ are highlighted as light blue ribbons in the corresponding regions of PEDF and ACT, respectively; the 4 amino acids substitutions between PEDFₚₑₚ and CHIMERAₚₑₚ are highlighted in dark blue and labeled in red; the numbering for both proteins begins at the secretory signal peptide; VEGF was injected in one eye, and VEGF+CHIMERAₚₑₚ in the contralateral eye; CHIMERAₚₑₚ (equimolar to PEDFₚₑₚ in FIG. 3a) had no discernible effect on VEGF-induced retinal vascular permeability by fluorescein angiogram (c) or by Evans blue assay (n=27) (D).

FIG. 5 illustrates that two activities of PEDF—inhibiting endothelial cell migration and inhibiting vasopermeability—require the same 4 amino acids wherein (a) VEGF₁₆₄-stimulated bovine retinal capillary endothelial cell migration was measured in the presence of various concentrations of PEDF, ACT, or HSP47. PEDF inhibited VEGF-induced migration in a dose-dependent manner with a Kd of 0.5 nM; ACT and HSP47 lacked this activity, the number of migrated cells in the presence of VEGF₁₆₄ minus the number in the absence of any added agent represents 100% maximal migration; each point represents the mean of quadruplicates ±SE; (b) VEGF₆₁₄-stimulated bovine retinal capillary endothelial cell migration was measured as in part a. PEDFₚₑₚ, but not ACTₚₑₚ or CHIMERAₚₑₚ, inhibited VEGF-stimulated bovine retinal capillary endothelial cell migration; PEDFₚₑₚ inhibited VEGF-stimulated endothelial cell migration (Kd=3 nM) to a similar extent as full length PEDF; ACTₚₑₚ and CHIMERAₚₑₚ had no effect on VEGF-stimulated endothelial cell migration.

FIG. 6 represents the full length PEDF amino acid sequence using the one letter code (SEQ ID NO.: 1).

FIG. 7 represents the amino acid sequence of the PEDF 44 AA peptide using the one letter code (SEQ ID NO.: 2).

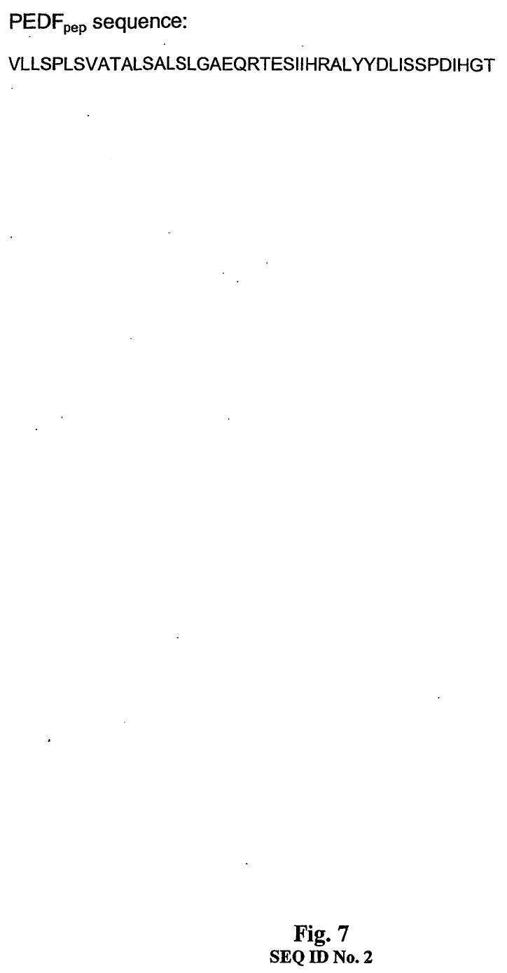

FIG. 8 represents the amino acid sequence of the PEDF 44 AA peptide using the one letter code (SEQ ID NO.: 3) with the glutamate at the 101 amino acid position, isoleucine at the 103 amino acid position, leucine at the 112 and serine at the 115 amino acid position underlined for illustration of their position within the 44 amino acid peptide of PEDF.

### DETAILED DESCRIPTION OF THE INVENTION

It is understood that this invention is not limited to the particular materials and methods described herein. It is also to be understood that the terminology used herein is for the purpose of describing particular embodiments and is not intended to limit the scope of the present invention which will be limited only by the appended claims. As used herein, the singular forms “a”, “an”, and “the” include plural reference unless the context clearly dictates otherwise. For example, a reference to “eye tissue” includes a plurality of cells known to those skilled in the art.

Unless defined otherwise, all technical and scientific terms used herein have the same meanings as commonly understood by one of ordinary skill in the art to which this invention belongs. All publications mentioned herein are cited for the purpose of describing and disclosing the permeability models, protocols, reagents and vectors which are reported in the publications and which might be used in connection with the invention. Nothing herein is to be construed as an admission that the invention is not entitled to antedate such disclosure by virtue of prior invention.

Relationship Between PEDF, VEGF and their Biological Activities

The relationships between the various activities of PEDF and VEGF are not entirely clear. Initial studies showed that PEDF induced neurite outgrowth 13, and VEGF promoted angiogenesis and vascular permeability^{1-3,33-35}. The report of PEDF's anti-angiogenic activity revealed an antagonistic relationship between PEDF and VEGF. In various types of neuronal cells, PEDF and VEGF share similar activities: both are neurotrophic and neuroprotective^{5,25,36}. Thus, VEGF has a triad of activities, (i) promoting angiogenesis, (ii) promoting neuronal survival and growth, and (iii) promoting vascular permeability

Vascular permeability plays a key pathophysiologic role not only in nonproliferative diabetic retinopathy, but also in many other disease states. The retinal vasculature is a preferred model system to study PEDF's potential effect on vascular permeability because the retinal vessels are easily observed through the clear optical system of the eye. In nonproliferative diabetic retinopathy, one of the most common causes of human visual loss, increased vascular permeability is the sine qua non of diabetic retinal edema. The gold standard diagnostic test for diabetic retinal edema is fluorescein angiography, a test used to demonstrate VEGF's central role in the pathophysiology of diabetic retinopathy 38. The mouse eye injected with VEGF has increased vascular permeability, resulting in increased fluorescein leakage. In the discovery of the present invention, this increase was counteracted when PEDF was co-injected, a finding confirmed with the quantitative Evans blue assay. Thus, PEDF, like VEGF, also possesses a triad of activities. PEDF not only functions as an anti-angiogenic and neurotrophic/neuroprotective agent, but also inhibits pathologically increased vascular permeability. Furthermore, PEDF is naturally present in the eye in significant quantities, and thus these activities may help maintain the normal physiology of the eye.

Since the neurotrophic/neuroprotective function of the triad of PEDF activities is likely receptor mediated^{24,25}, thus the anti-angiogenic and anti-vasopermeability activities are also likely to be receptor mediated. This was confirmed in that PEDF inhibits VEGF stimulated endothelial cell migration with an IC₅₀ of 0.5 nM and that PEDFₚₑₚ has an IC₅₀ of 3.0 nM, within the same order of magnitude as that of full length PEDF. The similar PEDF concentrations required for half-maximal neuronal or anti-angiogenic activities are consistent with the hypothesis that the neurotrophic/neuroprotective and anti-angiogenic activities share the same cell surface receptor. The localization of the active site of all 3 PEDF activities to the same 44-amino acid region (hereinafter referred to as “PEDF 44 AA peptide” and also referred to in the Brief description of the Figures and the Examples Sections as “PEDFₚₑₚ”, see SEQ ID NO.: 2) suggests that the activities are mediated by the same or similar receptors.

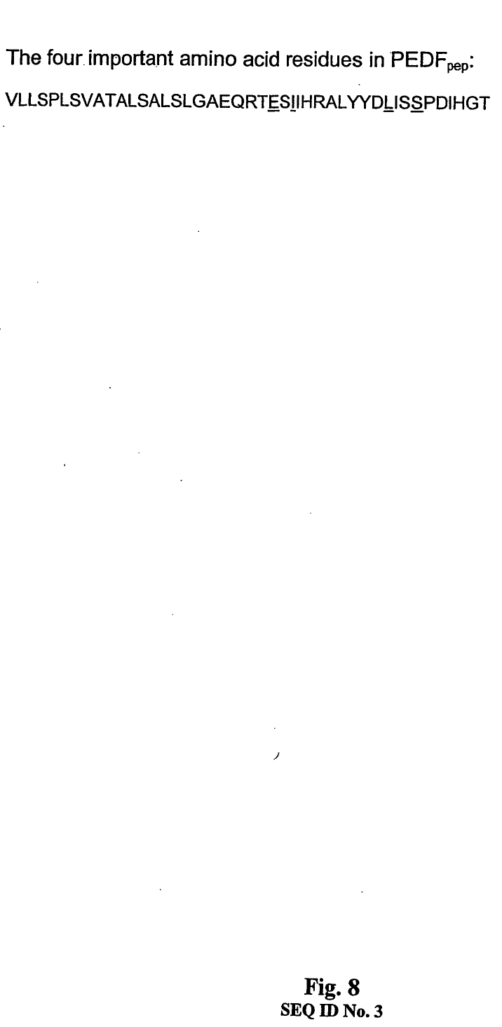

To further refine the localization of the active site within PEDF 44 AA peptide, a chimeric peptide, CHIMERAₚₑₚ was prepared with the hypothesis that the bioactivity would be abolished if the key amino acid residues in PEDF 44 AA peptide were substituted with corresponding residues from ACT or HSP47 (underlined in FIG. 8). When the 4 candidate amino acid residues were mutated in PEDF 44 AA peptide, bioactivity was lost. CHIMERAₚₑₚ identical to PEDF 44 AA peptide, with the exception that these 4 amino acid residues are substituted with the corresponding residues of either ACT or HSP47, is inactive in antagonizing any of VEGF's activities on the vascular system, in the endothelial cell migration assay, by fluorescein angiography, or by Evans blue assay.

In addition to the identification of the neurotrophic/neuroprotective region of PEDF within amino acid residues 78 to 121 (PEDF 44 AA peptide), a number of other binding sites on PEDF have been mapped: the acidic heparin binding domain; the collagen binding domain within β-sheet A strands 2 and 3 and helix F; and the serpin exposed loop at residues 367 to 387^{31,39}. In this invention, the anti-angiogenic and anti-vasopermeability active sites were localized to amino acid residues glutamate₁₀₁, isoleucine₁₀₃, leucine₁₁₂, and serine₁₁₅, indicating that the sites for these two activities are identical or extremely similar. These findings suggest that either a single receptor, or multiple receptors with very similar binding specificities serve these two or all 3 activities. An example of multiple receptors of distinct function, but with extremely similar binding specificities, is the 2 mannose-6-phosphate receptors⁴⁰⁻⁴²

### PEDF, PEDF 44 AA Peptide and Methods of Use:

The invention also encompasses the use of full length pigment epithelium-derived growth factor (PEDF; Steele et al., 1993, Proc. Natl. Acad. Sci. USA 90(4):1526-1530) and any derivative of PEDF for inhibiting vascular permeability, inhibiting angiogenesis and promoting neuroprotection, including, most particularly, PEDF 44 AA peptide and homologs thereof. The invention also encompasses the use of a nucleic acid encoding full length PEDF and any antiangiogenic or antivasopermeability derivative of PEDF, including, most particularly, PEDF 44 AA peptide and homologs thereof.

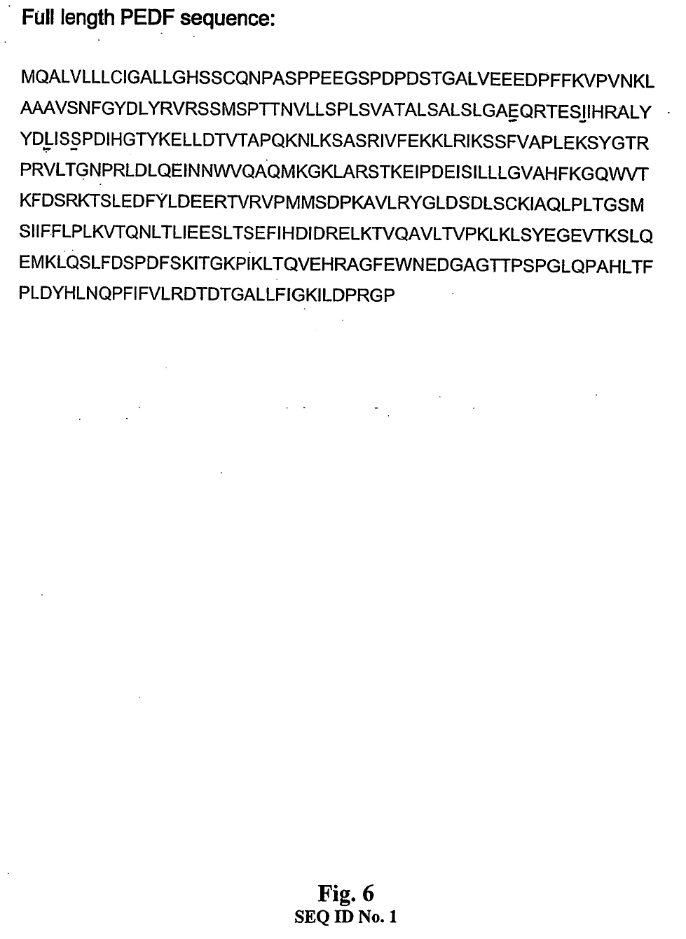

Within the context of the inventive method, PEDF is a protein having potent inhibitory activity on vascular permeability and angiogenesis. One form of PEDF polypeptide (full length PEDF) is set forth in FIG. 6 (SEQ ID NO: 1); however, the invention is not limited to the use of this exemplary sequence. Indeed, other PEDF sequences are known in the art (see, e.g., published international patent applications WO 95/33480 and WO 93/24529). Further, it is well known that genetic sequences can vary between different species and individuals. This natural scope of allelic variation is included within the scope of the present invention. Additionally and alternatively, a PEDF polypeptide can include one or more point mutations from the exemplary sequence or another naturally occurring PEDF polypeptide. Thus, a PEDF polypeptide is typically at least about 75% homologous to all or a portion of SEQ ID NO:1 and preferably is at least about 80% homologous to all or a portion of SEQ ID NO:1 (e.g., at least about 85% homologous to SEQ ID NO:1); more preferably the PEDF polypeptide is at least about 90% homologous to all or a portion of SEQ ID NO:1 (such as at least about 95% homologous to all or a portion of SEQ ID NO:1), and most preferably the PEDF polypeptide is at least about 97% homologous to all or a portion of SEQ ID NO:1. Indeed, the PEDF polypeptide can also include other domains, such as epitope tags and His tags (e.g., the protein can be a fusion protein).

Within the context of the present invention, a PEDF polypeptide or PEDF 44 AA peptide can be or comprise insertion, deletion, or substitution mutants of a known PEDF sequence or derivative thereof. Preferably, any substitution is conservative in that it minimally disrupts the biochemical properties of the PEDF polypeptide. Thus, where mutations are introduced to substitute amino acid residues, positively-charged residues (H, K, and R) preferably are substituted with positively-charged residues; negatively-charged residues (D and E) preferably are substituted with negatively-charged residues; neutral polar residues (C, G, N, Q, S, T, and Y) preferably are substituted with neutral polar residues; and neutral non-polar residues (A, F, I, L, M, P, V, and W) preferably are substituted with neutral non-polar residues. Moreover, the PEDF polypeptide can be an active fragment of a known PEDF protein or fragment thereof most preferably PEDF 44 AA peptide. Of course, while insertion, deletion, or substitution mutations can affect glycosylation of the protein, a PEDF polypeptide need not be glycosylated to possess the requisite inhibitory activities on vascular permeability and angiogenesis for use in the inventive method.

The invention should further be construed to include the use of a PEDF polypeptide or PEDF 44 AA peptide which may contain one or more D-isomer forms of the amino acids of PEDF. Production of a retro-inverso D-amino acid PEDF peptide where the peptide is made with the same amino acids as disclosed, but at least one amino acid, and perhaps all amino acids are D-amino acids is a simple matter once armed with the present invention. When all of the amino acids in the peptide are D-amino acids, and the N- and C-terminals of the molecule are reversed, the result is a molecule having the same structural groups being at the same positions as in the L-amino acid form of the molecule. However, the molecule is more stable to proteolytic degradation and is therefore useful in many of the applications recited herein.

The method of the invention should also be construed to include the use of PEDF or PEDF 44 AA peptide in the form of nucleic acid encoding biologically active PEDF, or any fragment thereof having PEDF biological activity, as defined herein. Thus the invention should be construed to include the use of nucleic acid, which encodes the fragments of PEDF and any derivatives thereof or a fragment thereof encoding biologically active PEDF.

By the term “biologically active PEDF” as used herein is meant any PEDF polypeptide, fragment or derivative, most particularly, PEDF 44 AA peptide which is capable of inhibiting vascular permeability and angiogenesis in any of the assays presented in the experimental details/examples section contained herein.

A biologically active fragment of PEDF is exemplified herein in the examples section as being a 44 amino acid fragment of PEDF (44 mer). The procedures for the isolation and characterization of this fragment are provided in detail herein in view of the state of skill in the art. Thus, it is an easy matter, following the directions provided herein, to identify this biologically active fragment of PEDF useful in the present invention, and the invention therefore must be construed to include any and all such homologs and any modifications and derivatives thereof, as disclosed herein. In addition, the invention should be construed to include any and all nucleic acids which encode biologically active fragments of PEDF as that term is defined herein. The term “PEDF” used in the claims appended hereto, should be construed to include all forms of biologically active PEDF as defined herein.

By the term “exogenous” as used herein to refer to PEDF or PEDF 44 AA peptide, the term should be construed to include any and all PEDF or PEDF 44 AA peptide which is not naturally expressed in a cell. For example, “exogenous PEDF” should be construed to include PEDF expressed from a nucleic acid which has been introduced into a cell using recombinant technology, PEDF which is added to a cell and any and all combinations thereof. Therefore, the term should not be construed to be limited solely to the addition of PEDF to a cell per se, but should be expanded to include the expression of PEDF in a cell when the PEDF is expressed from a nucleic acid which has been introduced into the cell.

PEDF polypeptides and PEDF 44 AA peptides inhibit vascular permeability, in part, by attenuating the transcellular vacuolar transport and/or fenestration, and/or by preservation of tight intercellular junctions in endothelial cells. Thus, the invention provides a method of inhibiting vacuolar transport, fenestration, or of promoting tight junctions by providing exogenous PEDF or PEDF 44 AA peptide to such cells. Aside from attenuating vasopermeability, the method is useful for treating disorders associated with stimulation of vascular permeability in the eye such as cystoid macular edema, uveitic retinal edema, vascular occlusive diseases. In other organ systems, the method is useful in cerebral, pulmonary, bowel edema, and other exudative pathologies.

PEDF polypeptides and PEDF 44 AA peptides inhibit angiogenesis, in part, by attenuating the migration and/or contraction of activated endothelial cells, thus reducing the ability of endothelia to expand within the tissue. Thus, the invention provides a method of inhibiting endothelial cell migration and expansion by providing exogenous PEDF or PEDF 44 AA peptide to such cells. Aside from attenuating angiogenesis, the method is useful for treating disorders associated with stimulation of endothelial cell migration such as intestinal adhesions, Crohn's disease, atherosclerosis, scleroderma and rheumatoid arthritis.

In accordance with the inventive method, PEDF or PEDF 44 AA peptide is provided to endothelial cells associated with the tissue of interest. Such cells can be cells comprising the tissue of interest, exogenous cells introduced into the tissue, or neighboring cells not within the tissue. Thus, for example, the cells can be cells of the tissue, and PEDF or PEDF 44 AA peptide is provided to them in situ such that the PEDF or PEDF 44 AA peptide contacts the cells. Alternatively, the cells can be cells introduced into the tissue, in which case the PEDF or PEDF 44 AA peptide can be transferred to the cells before they are so introduced into the tissue (e.g., in vitro), as well as being transferred in situ after introduction into the tissue.

When PEDF or PEDF 44 AA peptide is introduced into cells which are then transferred to the mammal, the invention should not be construed as being limited by the manner in which PEDF or PEDF 44 AA peptide is introduced into the cells. Nor should the invention be construed to be limited to the manner in which the cells are introduced to the mammal. As described in more detail below, methods of introducing DNA into cells are well known as are methods or delivering such cells to a tissue in a mammal.

The tissue with which the endothelial cells are associated is any tissue in which it is desired to inhibit the migration or expansion of endothelia, (e.g., for inhibiting angiogenesis), and to inhibit vacuolar transport, Penetration, or leakage across tight junctions (e.g. for inhibiting vasopermeability). In one application, the tissue can be eye tissue, in which case the presence of exogenous PEDF or PEDF 44 AA peptide will inhibit novel vascular permeability and angiogenesis associated with a variety of disorders of the eye. For example, the inventive method is useful for treating eye injury, hypoxia, infection, surgery, laser surgery, diabetes, retinoblastoma, macular degeneration, ischemic retinopathy, or other diseases or disorders of the eye. In this respect, the method is useful for restoring vision, preventing blindness or retarding loss of vision associated with a variety of eye diseases. The vast majority of diabetic patients eventually suffer vision impairment due to overgrowth of vessels in the retina in response to ischemia caused by the disease. Similarly, premature infants exposed to high levels of oxygen develop retinopathy as a result of retinal vein occlusion or other vascular or ischemic abnormalities. As described herein, ischemic-induced retinopathies may be prevented and or treated with by systemic or local administration of PEDF or PEDF 44 AA peptide. In the case of laser surgery, with respect to the eye, PEDF or PEDF 44 AA peptide may be used to prevent the re-growth of vessels after treatment. Lasers are used to abate excessive vessels, but they also ablate retina with vision potential, and create a wound in the retina that induces some angiogenesis. Systemic or local treatment with PEDF or PEDF 44 AA peptide should serve to prevent such re-growth and retain viable retinal tissue which otherwise would be ablated

Gene therapy can be achieved to deliver PEDF or PEDF 44 AA peptide by constructing retroviral gene transfer vectors using the methods of U.S. Pat. No. 5,614,404, describing recombinant viral vectors which coexpress heterologous polypeptides capable of assembling into defective nonself-propagating viral particles. Viruses useful as gene transfer vectors include retrovirus, which are the vectors most commonly used in human clinical trials. To generate a gene therapy vector, the gene of interest is cloned into a replication-defective retroviral plasmid which contains two long terminal repeats (LTR), a primer binding site, a packaging signal, and a polypurine tract essential to reverse transcription and the integration functions of retrovirus after infection. To produce viral vector, the plasmid form of a vector is transfected into a packaging cell line which produces Gag, Pol and Env of the retroviral structural proteins required for particle assembly. A producer cell line is usually generated using a selective marker, often a G418 resistant gene carried by the retroviral vector. The resulting cell line can be encapsulated, as described in PCT International patent application WO 97/44065, which describes biocompatible capsules containing living packaging cells that secrete a viral vector for infection of a target cell, and methods of delivery for an advantageous infectivity of the target cells.

By the term “retinopathy” as used herein, is meant the abnormal development of blood vessels within or around the retina that may or may not enter the vitreous. Injury, disease, ischemic events, laser or other iatrogenic treatments may induce retinopathy.

In other embodiments, the tissue is a tumor (e.g., a benign or cancerous growth), in which case the inventive method will inhibit the growth of blood vessels within and to the tumor, and in some cases, induce tumor cells to differentiate and thus divide slowly. Inhibiting the growth of blood vessels within tumors prevents sufficient nutrients and oxygen from being supplied to the tumor to support growth beyond a given size. Thus, the inventive method can prevent the nucleation of tumors from cancerous cells already present due to genetic predisposition (e.g., BRCA-1 mutation carriers, Li Fraumeni patients with p53 mutations, etc.) or the presence of external carcinogens (e.g., tobacco, alcohol, industrial solvents, etc.). Aside from preventing tumorigenesis, the inventive method can retard the growth of existing tumors, thus rendering them more easily contained and excised and may cause them to regress. This application is highly advantageous for treating tumors that are difficult to operate on (e.g., brain or prostate tumors). In addition, the method is useful for treatment of childhood tumors, including, but not limited to, neuroblastoma. Moreover, minimizing the number of blood vessels within existing tumors lessens the probability that the tumor will metastasize. In treating tumors, the method can be used alone or in conjunction with other treatments, to control the growth of tumors. Indeed, employing the inventive method can potentiate the response of some tumors to other therapies. For example, the inventive method optionally can be employed as a pretreatment for (e.g., for about a week in advance of), and continued during, a chemotherapeutic or radiation regimen. The method of the invention may also be used in conjunction with the use of biological response modifiers, such as for example, interferon, or other anti-angiogenic agents, and also is useful in conjunction with the use of agents which induce the production of anti-angiogenic agents in vivo. Further, the method of the invention may be used in conjunction with agents which promote the differentiation of cells, particularly, but not limited to agents which promote the differentiation of brain tumor cells.

Where the inventive method is applied to other tissues, the prevention of neovascularization effectively treats a host of disorders. Thus, for example, the inventive method can be used as part of a treatment for disorders of blood vessels (e.g., hemangiomas and capillary proliferation within atherosclerotic plaques), muscle (e.g., myocardial angiogenesis or angiogenesis within smooth muscles), joints (e.g., arthritis, hemophiliac joints, etc.), and other disorders associated with angiogenesis (e.g., Osler-Webber Syndrome, plaque neovascularization, telangiectasia, angiofibroma, wound granularization, etc.). In addition, the invention is useful for treatment of nasal polyps, especially in cystic fibrosis patients, leukemia which stems from bone marrow cell abnormal growth, and prostate cancer. The invention can be construed in general to be useful for treatment of benign neoplasias.

The inventive method is also useful as a means of preventing the occurrence of a disease or disorder associated with vascular permeability or angiogenesis, i.e., the methods are useful as prophylactic methods for the prevention of disease in patients at risk for the disease. For example, and without limitation, PEDF or PEDF 44 AA peptide may be used to prevent the onset of diabetic retinopathy in a patient having diabetes, to prevent the onset of cancer in persons known to be at risk for certain cancers, and the like. Thus, the methods of the invention should not be construed as being limited to treatment of overt disease, but rather, should be construed as being useful for the prevention of disease in patients who are at risk.

The invention should also be construed to include treatment of precancerous lesions, for example, but without limitation, nasal polyps, particularly in patients having cystic fibrosis. Nasal polyps in these patients are angiogenic, and further, the cerebral spinal fluid of cystic fibrosis patients contains an excess of the angiogenic factor VEGF. Alleviation of these conditions, especially in cystic fibrosis patients, wherein the alleviation comprises administration of PEDF or PEDF 44 AA peptide is therefore included in the present invention.

Within the context of the inventive method, PEDF or PEDF 44 AA peptide can be supplied alone or in conjunction with other known antiangiogenic factors. For example, PEDF or PEDF 44 AA peptide can be used in conjunction with antibodies and peptides that block integrin engagement, proteins and small molecules that inhibit metalloproteinases (e.g., marmistat), agents that block phosphorylation cascades within endothelial cells (e.g., herbamycin), dominant negative receptors for known inducers of angiogenesis, antibodies against inducers of angiogenesis or other compounds that block their activity (e.g., suramin), or other compounds (e.g., retinoids, IL-4, interferons, etc.) acting by other means. Indeed, as such factors modulate angiogenesis by different mechanisms, employing PEDF or PEDF 44 AA peptide in combination with other antiangiogenic agents can potentiate a more potent (and potentially synergistic) inhibition of angiogenesis within the desired tissue. PEDF or PEDF 44 AA peptide can be used with one or more other antiangiogenic factors. Preferably, at least two antiangiogenic factors may be used in conjunction with PEDF or PEDF 44 AA peptide.

As discussed herein, PEDF or PEDF 44 AA peptide is a proteinaceous factor. Thus, in one protocol, the method involves providing PEDF or PEDF 44 AA peptide by supplying a PEDF polypeptide or PEDF 44 AA peptide to the cells (e.g., within a suitable composition). Any suitable method can be employed to obtain a PEDF polypeptide or PEDF 44 AA peptide for use in the present invention. Many suitable PEDF polypeptides can be purified from tissues which naturally produce PEDF or from media conditioned by a variety of PEDF-producing cells (e.g., retinoblastoma cell line WER127). For example, it is known that PEDF is produced by all types of muscle, megakaryocytes of the spleen, fibroblasts, kidney tubules, cerebellar Purkinje cells, piliosebaceous glands of hair follicles, and retinal cells. A particularly good source of naturally occurring PEDF is the vitreous and aqueous humors of the eye.

One protocol for purifying PEDF from protein extracts of these (or other sources) is by concentration/dialysis using a 30 kDa ultrafiltration membrane followed by protein precipitation in a range of about 65% to about 95% ammonium sulfate, followed by a lentil lectin sepharose column at 0.5 M methyl-.alpha.-D-mannopyranoside, followed by gradient/isocratic elution at 0.5 M NaCl from a PHARMACIA HiTrap heparin column. Other protocols for purifying PEDF polypeptides are known in the art (see, e.g., published international patent applications WO 95/33480 and WO 93/24529). The native PEDF polypeptide represented by SEQ ID NO:1 is identified via SDS-PAGE as a protein of about 45 to 50 kDa. Other PEDF polypeptides or PEDF 44 AA peptide can be synthesized using standard direct peptide synthesizing techniques (e.g., as summarized in Bodanszky, 1984, Principles of Peptide Synthesis (Springer-Verlag, Heidelberg), such as via solid-phase synthesis (see, e.g., Merrifield, 1963, J. Am. Chem. Soc. 85:2149-2154; Barany et al., 1987, Int. J. Peptide Protein Res. 30:705-739; and U.S. Pat. No. 5,424,398). Of course, as genes for PEDF polypeptides are known (see, e.g., published international patent applications WO 95/33480 and WO 93/24529); see also GenBank accession no. U29953), or can be deduced from the polypeptide sequences discussed herein, a PEDF polypeptide or PEDF 44 AA peptide can be produced by standard recombinant DNA methods.

In other protocols, PEDF polypeptide or PEDF 44 AA peptide can be provided to the tissue of interest by transferring an expression vector including a nucleic acid encoding PEDF to cells associated with the tissue of interest. The cells produce and secrete the PEDF polypeptide such that it is suitably provided to endothelial cells within the tissue to inhibit their contraction or migration (for angiogenesis) and fenetration, vacuolar or transjunctional transport (for vasopermeability) and, thus, to attenuate vascular permeability and angiogenesis within the tissue of interest or systemically. Nucleic acid sequences which encode PEDF polypeptides are known (see, e.g., published international patent applications WO 95/33480 and WO 93/24529); see also GenBank accession no. U29953), and others can be deduced from the polypeptide sequences discussed herein. Thus, PEDF or PEDF 44 AA peptide expression vectors typically include isolated nucleic acid sequence which are homologous to PEDF or PEDF 44 AA peptide sequences, e.g., they will hybridize to at least a fragment of the known sequences under at least mild stringency conditions, more preferably under moderate stringency conditions, most preferably under high stringency conditions (employing the definitions of mild, moderate, and high stringency as set forth in Sambrook et al., 1989, Molecular Cloning: A Laboratory Manual, 2d edition, Cold Spring Harbor Press).

In addition to the nucleic acid encoding PEDF or PEDF 44 AA peptide, an expression vector includes a promoter, and, in the context of the present invention, the promoter must be able to drive the expression of the PEDF or PEDF 44 AA peptide cDNA within the cells. Many viral promoters are appropriate for use in such an expression cassette (e.g., retroviral ITRs, LTRs, immediate early viral promoters (IEp) (such as herpesvirus IEp (e.g., ICP4-IEp and ICP0-IEp) and cytomegalovirus (CMV) IEp), and other viral promoters (e.g., late viral promoters, latency-active promoters (LAPs), Rous Sarcoma Virus (RSV) promoters, and Murine Leukemia Virus (MLV) promoters)). Other suitable promoters are eukaryotic promoters which contain enhancer sequences (e.g., the rabbit .beta.-globin regulatory elements), constitutively active promoters (e.g., the P-actin promoter, etc.), signal and/or tissue specific promoters (e.g., inducible and/or repressible promoters, such as a promoter responsive to TNF or RU486, the metallothionine promoter, etc.), and tumor-specific promoters.

Within the expression vector, the PEDF or PEDF 44 AA peptide cDNA and the promoter are operably linked such that the promoter is able to drive the expression of the PEDF or PEDF 44 AA peptide gene. Furthermore, the expression vector can optionally include other elements, such as splice sites, polyadenylation sequences, transcriptional regulatory elements (e.g., enhancers, silencers, etc.), or other sequences.

The expression vector must be introduced into the cells in a manner such that they are capable of expressing the isolated nucleic acid encoding PEDF or PEDF 44 AA peptide contained therein. Any suitable vector can be so employed, many of which are known in the art. Examples of such vectors include naked DNA vectors (such as oligonucleotides or plasmids), viral vectors such as adeno-associated viral vectors (Berns et al., 1995, Ann. N.Y. Acad. Sci. 772:95-104), adenoviral vectors (Bain et al., 1994, Gene Therapy 1:S68), herpesvirus vectors (Fink et al., 1996, Ann. Rev. Neurosci. 19:265-287), packaged amplicons (Federoff et al., 1992, Proc. Natl.

Acad. Sci. USA 89:1636-1640), papilloma virus vectors, picornavirus vectors, polyoma virus vectors, retroviral vectors, SV40 viral vectors, vaccinia virus vectors, and other vectors. In addition to the expression vector of interest, the vector can also include other genetic elements, such as, for example, genes encoding a selectable marker (e.g., .beta.-gal or a marker conferring resistance to a toxin), a pharmacologically active protein, a transcription factor, or other biologically active substance.

Any vector selected must be capable of being produced in large quantities in eukaryotic cells. In addition, it is necessary that the vector can be constructed such that it is capable of being transferred into the cells of interest either with or without PEDF or PEDF 44 AA peptide sequence, such that the vector which does not contain PEDF or PEDF 44 AA peptide sequences serves as a control vector, and that the vector which includes PEDF or PEDF 44 AA peptide sequences is the experimental or therapeutic vector. Methods for manipulating the vector nucleic acid are well known in the art (see, e.g., Sambrook et al., supra) and include direct cloning, site specific recombination using recombinases, homologous recombination, and other suitable methods of constructing a recombinant vector. In this manner, an expression vector can be constructed such that it can be replicated in any desired cell, expressed in any desired cell, and can even become integrated into the genome of any desired cell.

The PEDF or PEDF 44 AA peptide expression vector is introduced into the cells by any means appropriate for the transfer of DNA into cells. Many such methods are well-known in the art (Sambrook et al., supra; see also Watson et al., 1992, Recombinant DNA, Chapter 12, 2d edition, Scientific American Books). Thus, plasmids are transferred by methods such as calcium phosphate precipitation, electroporation, liposome-mediated transfection, gene gun, microinjection, viral capsid-mediated transfer, polybrene-mediated transfer, protoplast fusion, etc. Viral vectors are best transferred into cells by direct infection of the cells. However, the mode of infection may vary depending on the exact nature of the virus and the cell.

Cells into which the PEDF or PEDF 44 AA peptide cDNA has been transferred under the control of an inducible promoter if necessary, can be used in the inventive method as transient transformants. Such cells themselves may then be transferred into a mammal for therapeutic benefit therein. Typically, the cells are transferred to a site in the mammal such that PEDF expressed therein and secreted therefrom contacts the desired endothelial cells in order that vascular permeability or angiogenesis is inhibited. Alternatively, particularly in the case of cells to which the vector has been added in vitro, the cells may first be subjected to several rounds of clonal selection (facilitated usually by the use of a selectable marker sequence in the vector) to select for stable transformants. Such stable transformants are then transferred to a mammal for therapeutic benefit therein.

The PEDF or PEDF 44 AA peptide may also be provided to the endothelial cells by transfecting into a population of other cells a vector comprising an isolated nucleic acid encoding PEDF or PEDF 44 AA peptide, whereby the PEDF or PEDF 44 AA peptide is expressed in and secreted from said other cells. The population of other cells so transfected is then transferred to a site in the mammal where PEDF or PEDF 44 AA peptide so secreted contacts the endothelial cells and inhibits vascular permeability or angiogenesis. Expression and secretion of PEDF or PEDF 44 AA peptide from the other cells then has benefit on the endothelial cells. It is not necessary that the DNA encoding PEDF or PEDF 44 AA peptide be stably integrated into the cells. PEDF or PEDF 44 AA peptide may be expressed and secreted from non-integrated or from integrated DNA in a cell.

Within the cells, the PEDF or PEDF 44 AA peptide construct is expressed such that the cells express and secrete the PEDF polypeptide or PEDF 44 AA peptide. Successful expression of the gene can be assessed using standard molecular biological techniques (e.g., Northern hybridization, Western blotting, immunoprecipitation, enzyme immunoassay, etc.).

Depending on the location of the tissue of interest, PEDF can be supplied in any manner suitable for the provision of PEDF to endothelial cells within the tissue of interest. Thus, for example, a composition containing a source of PEDF (i.e., a PEDF polypeptide or a PEDF expression vector, or cells expressing PEDF, as described herein) can be introduced into the systemic circulation, which will distribute the source of PEDF to the tissue of interest. Alternatively, a composition containing a source of PEDF can be applied topically to the tissue of interest (e.g., injected, or pumped as a continuous infusion, or as a bolus within a tumor or intercutaneous or subcutaneous site, dropped onto the surface of the eye, etc.).

Where the source of PEDF or PEDF 44 AA peptide is a PEDF polypeptide (e.g., within a suitable composition), it is provided in a concentration and for a time sufficient to inhibit vascular permeability or angiogenesis within the tissue.

To facilitate the inventive method, the invention provides a pharmacological composition comprising a source of PEDF or PEDF 44 AA peptide and a suitable diluent. In addition to the source of PEDF or PEDF 44 AA peptide, the composition includes a diluent, which includes one or more pharmacologically-acceptable carriers. Pharmaceutical compositions for use in accordance with the present invention can be formulated in a conventional manner using one or more pharmacologically or physiologically acceptable carriers comprising excipients, as well as optional auxiliaries which facilitate processing of the active compounds into preparations which can be used pharmaceutically. Proper formulation is dependent upon the route of administration chosen. Thus, for systemic injection, the source of PEDF or PEDF 44 AA peptide can be formulated in aqueous solutions, preferably in physiologically compatible buffers that may, if needed, contain stabilizers such as polyethylene glycol. For transmucosal administration, penetrants appropriate to the barrier to be permeated are used in the formulation. Such penetrants are generally known in the art. For oral administration, the source of PEDF or PEDF 44 AA peptide can be combined with carriers suitable for inclusion into tablets, pills, dragees, capsules, liquids, gels, syrups, slurries, liposomes, suspensions and the like. For administration by inhalation, the source of PEDF or PEDF 44 AA peptide is conveniently delivered in the form of an aerosol spray presentation from pressurized packs or a nebulizer, with the use of a suitable propellant. The source of PEDF or PEDF 44 AA peptide can be formulated for parenteral administration by injection, e.g., by bolus injection or continuous infusion. Such compositions can take such forms as suspensions, solutions or emulsions in oily or aqueous vehicles, and can contain formulatory agents such as suspending, stabilizing and/or dispersing agents. For application to the skin, the source of PEDF or PEDF 44 AA peptide can be formulated into a suitable gel, magma, creme, ointment, or other carrier. For application to the eyes, the source of PEDF or PEDF 44 AA peptide can be formulated in aqueous solutions, preferably in physiologically compatible buffers, in addition to the methods described for the skin. The source of PEDF or PEDF 44 AA peptide can also be formulated into other pharmaceutical compositions such as those known in the art. A detailed discussion of pharmaceutical compositions and formulations is provided elsewhere herein.

In addition to all of the above, the invention should also be construed to include methods of regulating the expression of endogenous PEDF in a cell. For example, it is possible to upregulate PEDF production in a cell by inducing transient hyperoxia in the cell. Such treatment has the added benefit of down-regulating inducers of angiogenesis. The invention should be construed to include the application of this method to each of the treatment modalities described herein.

### DEFINITIONS

As used herein, each of the following terms has the meaning associated with it in this section.

As used herein, the term “adjacent” is used to refer to nucleotide sequences which are directly attached to one another, having no intervening nucleotides. By way of example, the pentanucleotide 5′-AAAAA-3′ is adjacent the trinucleotide 5′-TTT-3′ when the two are connected thus: 5′-AAAAATTT-3′ or 5′-TTTAAAAA-3′, but not when the two are connected thus: 5′-AAAAACTTT-3′.

As used herein, “alleviating a symptom” means reducing the severity of the symptom.

As used herein, amino acids are represented by the full name thereof, by the three letter code corresponding thereto, or by the one-letter code corresponding thereto, as indicated in the following table:

Full Name Three-Letter Code/One-Letter Amino Acid Code: Aspartic Acid Asp D Glutamic Acid/Glutamate Glu E Lysine Lys K Arginine Arg R Histidine His H Tyrosine Tyr Y Cysteine Cys C Asparagine Asn N Glutamine Gln Q Serine Ser S Threonine Thr T Glycine Gly G Alanine Ala A Valine Val V Leucine Leu L Isoleucine Ile I Methionine Met M Proline Pro P Phenylalanine Phe F Tryptophan Trp W

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

An “mRNA-coding region” of a gene consists of the nucleotide residues of the coding strand of the gene and the nucleotide residues of the non-coding strand of the gene which are homologous with or complementary to, respectively, an mRNA molecule which is produced by transcription of the gene. It is understood that, owing to mRNA processing which occurs in certain instances in eukaryotic cells, the mRNA-coding region of a gene may comprise a single region or a plurality of regions separated from one another in the gene as it occurs in the genome. Where the mRNA-coding region of a gene comprises separate regions in a genome, “mRNA-coding region” refers both individually and collectively to each of these regions.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs).

A “condition” is a state of health of an animal wherein the animal cannot maintain homeostasis, and wherein if the disease is not ameliorated then the animal's health continues to deteriorate. A disease is “alleviated” if the severity of a symptom of the disease, the frequency with which such a symptom is experienced by a patient, or both, are reduced.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5 and 3′TATGGC share 50% homology.

As used herein, “homology” is used synonymously with “identity.”

The determination of percent identity between two nucledtide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator “http://www.ncbi.nlm.nih.gov/BLAST/”. BLAST nucleotide searches can be performed with the NBLAST program (designated “blastn” at the NCBI web site), using the following parameters: gap penalty=5; gap extension penalty-2; mismatch penalty-3; match reward=1; expectation value 10.0; and word size=1 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used. See http://www.ncbi.nlm.nih.gov.

The percent identity between two sequences can be determined using techniques similar to those described above, with or without allowing gaps. In calculating percent identity, typically exact matches are counted.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

A “polynucleotide” means a single strand or parallel and anti-parallel strands of a nucleic acid. Thus, a polynucleotide may be either a single-stranded or a double-stranded nucleic acid.

The term “nucleic acid” typically refers to large polynucleotides.

The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A first oligonucleotide anneals with a second oligonucleotide “with high stringency” if the two oligonucleotides anneal under conditions whereby only oligonucleotides which are at least about 75%, and preferably at least about 90% or at least about 95%, complementary anneal with one another. The stringency of conditions used to anneal two oligonucleotides is a function of, among other factors, temperature, ionic strength of the annealing medium, the incubation period, the length of the oligonucleotides, the GC content of the oligonucleotides, and the expected degree of non-homology between the two oligonucleotides, if known. Methods of adjusting the stringency of annealing conditions are known (see, e.g. Sambrook et al., 1989, Molecular Cloning: A Laboratory Manual, Cold Spring Harbor Laboratory, New York).

A “prophylactic” treatment is a treatment administered to a subject who does not exhibit signs of a disease or exhibits only early signs of the disease for the purpose of decreasing the risk of developing pathology associated with the disease.

A “therapeutic” treatment is a treatment administered to a subject, who exhibits signs of pathology for the purpose of diminishing or eliminating those signs.

A “therapeutically effective amount” of a compound is that amount of compound which is sufficient to provide a beneficial effect to the subject to which the compound is administered. A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### Modification and Synthesis of Peptides:

The following section refers to the modification of peptides and to their synthesis. It will be appreciated, of course, that the peptides useful in the methods of the invention may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C.sub.1-C.sub.5 branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal residue. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (-—NH.sub.2), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the biological activity of the peptide and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the methods of the invention.

The present invention also provides for analogs of proteins or peptides encoded by the nucleic acid disclosed herein. Analogs can differ from naturally occurring proteins or peptides by conservative amino acid sequence differences or by modifications which do not affect sequence, or by both.

For example, conservative amino acid changes may be made, which although they alter the primary sequence of the protein or peptide, do not normally alter its function. Conservative amino acid substitutions typically include substitutions within the following groups:

glycine, alanine;
valine, isoleucine, leucine;
aspartic acid, glutamic acid
asparagine, glutamine;
serine, threonine;
lysine, arginine;
phenylalanine, tyrosine.

As noted above, modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

The peptides of the present invention may be readily prepared by standard, well-established solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the alpha.-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method which utilized tert-butyloxcarbonyl as the a-amino protecting group, and the FMOC method which utilizes 9-fluorenylmethyloxcarbonyl to protect the .alpha.-amino of the amino acid residues, both methods of which are well-known by those of skill in the art. Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon BF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl blocking group at the N-terminus, for instance, the resincoupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

Prior to its use in the methods of the invention, the peptide is purified to remove contaminants. In this regard, it will be appreciated that the peptide will be purified so as to meet the standards set out by the appropriate regulatory agencies.

Any one of a number of a conventional purification procedures may be used to attain the required level of purity including, for example, reversed-phase high-pressure liquid chromatography (HPLC) using an alkylated silica column such as C.sub.4-, C.sub.8- or C.sub.18-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

Assays for Identifying Candidate Agents having PEDF Biological Activity:

The term “agent” or “compound” as used herein describes any molecule, e.g. protein or pharmaceutical, with the capability of mimicking or effectuating the biological action of PEDF. Generally a plurality of assay mixtures can be run in parallel with different agent concentrations to obtain a differential response to the various concentrations. Typically, one of these concentrations serves as a negative control, i.e. at zero concentration or below the level of detection.

Candidate agents (compounds) encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups.

The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Screening may be directed to known pharmacologically active compounds and chemical analogs thereof.

Where the screening assay is a binding assay utilizing the PEDF receptor (see U.S. Provisional Application 60/493,713, filed Aug. 7, 2003), hereby incorporated by reference in its entirety, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40.degree. C. Incubation periods are selected for optimum activity, but may also be optimized to facilitate rapid high-throughput screening.

### Pharmaceutical Compositions:

Compounds which are identified using any of the methods described herein may be formulated and administered to a mammal for treatment of the diseases disclosed herein are now described.

The invention encompasses the preparation and use of pharmaceutical compositions comprising a compound useful in the methods of the invention as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier”. means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys. Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e. about 20.degree. C.) and which is liquid at the rectal temperature of the subject (i.e. about 37.degree. C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or gel or cream or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65.degree. F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other ophthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsfying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Sustained release compositions comprising PEDF may be particularly useful. For example, sustained release compositions may be used in the vitrous and may also be used behind the eye. As stated elsewhere herein, sustained release compositions may also be useful in systemic or other delivery routes for administration of PEDF. One of ordinary skill in the art will know the appropriate sustained release compositions which can be used to treat the desired disease to achieve the desired outcome.

Typically dosages of the compound of the invention which may be administered to an animal, preferably a human, range in amount from 1.mu.g to about 100 g per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even lees frequently, such as once every several months or even once a year or less. The frequency of the dose will be readily apparent to the skilled artisan and will depend upon any number of factors, such as, but not limited to, the type and severity of the disease being treated, the type and age of the animal, etc.

### EXAMPLES

This invention is further illustrated by the following examples which are provided for the purpose of illustration only and the invention should in no way be construed as being limited to these examples but rather should be construed to encompass any and all variations which become evident as a result of the teaching provided herein. The contents of all references, patents, and published patent applications cited throughout this application, as well as the figures, are incorporated herein by reference.

### Exemplification

The following methods and materials were used in the following Examples:

Preparation of PEDF. Recombinant human PEDF was produced in human embryonic kidney carcinoma 293 cells as described¹⁹. PEDF protein was purified from the conditioned media according to previously described procedures⁴³. From a Mono S FPLC column, PEDF was eluted with a linear NaCl gradient (20 mM NaH₂PO₄, pH 6.2, 0 to 500 mM NaCl, 10% glycerol).

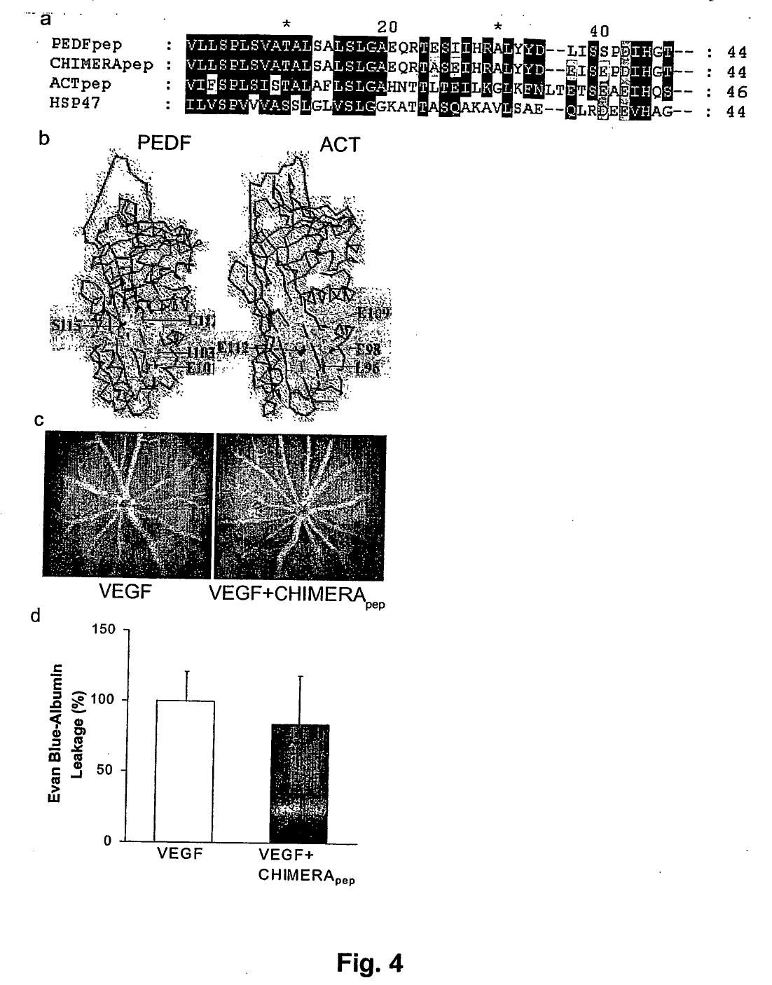

Preparation of synthetic peptides. Three peptides (FIG. 4a) were synthesized. The PEDF peptide (PEDFₚₑₚ) corresponded to amino acid residues 78-121 of the protein (GenBank™ accession number P36955). The ACT peptide (ACTₚₑₚ) corresponded to residues 73-118 of the protein (accession number P01011). A chimeric peptide (CHIMERAₚₑₚ), was 44 amino acids in length, with 40 amino acid residues from PEDF plus 4 amino acid residues from ACT or HSP47 (accession number P29043).

Intravitreal injection to assess bioactivity on vascular permeability. C57BL/6J mice, 6-8 weeks of age, were cared for in accordance with the Association for Research in Vision and Opthalmology Statement for the Use of Animals in Ophthalmic and Vision Research. For anesthesia, each received intramuscularly 20 mg/kg ketamine, 20 mg/kg xylazine and 800 mg/kg urethane in 0.3-0.4 ml of phosphate buffered saline (PBS, 1.06 mM KH₂PO₄, 0.15 M NaCl and 3.00 mM Na₂HPO₄, pH 7.4). Under 10× magnification, 1 μl of murine VEGF₁₆₄ (12.6 ng/μl in PBS; R&D Systems, Minneapolis, Minn.) was delivered through a 20° beveled glass pipette, with a tip diameter of 13-20 μm. The contralateral eye received an equal volume of PBS alone or PBS containing 12.6 ng VEGF₁₆₄, and a 20-fold molar excess of PEDF (232 ng), ACT (278 ng), HSP47 (278 ng), PEDFₚₑₚ (28.1 ng), ACTₚₑₚ (29.7 ng), or CHIMERAₚₑₚ (28.2 ng).

Fluorescein angiograghy. Twenty hours after intravitreal injections, each pupil was dilated with one drop of 1% atropine sulfate. After intraperitoneal injection of 0.1 ml of 25% fluorescein, successive retina photographs were taken with a Kowa Genesis camera. The first photograph was taken within 20 seconds of the intraperitoneal fluorescein injection. Time elapsed between the alternating right and left eye retinal photographs averages 10 seconds. Fluorescein leakage manifests as indistinct vascular borders progressing to diffusely hazy fluorescence.

Evans Blue assay. We used a modification of the method described by Qaum et al.⁴⁴ Briefly, each mouse received intravitreal injections of proteins or peptides, and intrajugular injection of Evans blue⁴⁴. After 2 hours, 200 μl blood was taken and assayed for Evans blue. The retina was extruded and dissected free from any vitreous or adherent retinal pigment epithelium.

To assess the Evans blue-albumin concentration, the optical density of the retinal extract and plasma samples were measured at 620 nm and 740 nm. The retinal vasopermeability was calculated as the quantity of retinal Evans blue normalized to retinal dry weight, plasma Evans blue concentration, and circulation time by using the formula as described^{32,44}. Since all animals in this report had 1 eye injected with VEGF alone, the retinal permeability in the VEGF injected eyes was normalized to the VEGF injected eye in the set of animals where 1 eye received PBS. The VEGF-induced increase in permeability was taken to be 100%.

BRCEC migration assay. Bovine retinal capillary endothelial cells (BRCEC) were isolated and cultured as described 45. After treatment with 1,1′-dioctadecyl-3,3,3′,3′-tetramethyl-indocarbocyanine perchlorate labeled acetylated low density lipoprotein (Dil-Ac-LDL; Biomedical Technologies Inc., Stoughton, Mass.), BRCEC were further purified by fluorescent cell sorter. Cells, between the fifth and ninth passage, were starved overnight in MEM with D-Val and 2% fetal bovine serum. Polycarbonate filters (10 μm pore-size, PVPF; Osmonics Inc., Minnetonka, Minn.) were coated with 100 μg/ml collagen. Quadruplicates with test samples in MEM D-Val (28 μl), and 10⁴ cells in MEM D-Val (50 μl) were placed in the lower and upper wells, respectively, of the microchemotaxis chamber (NeuroProbe, Gaithersburg, Md.). After incubating 8 hours at 37° C., the non-migrated cells on the upper surface of the filter were removed, and the filters were stained with Harris' hematoxylin. For each of the test samples, one 400× field per quadrant of each of the quadruplicates was counted. From the total cell number in the 4 fields, the mean and standard error of the quadruplicate wells were calculated. Baseline migration equaled the number of migrated cells with MEM D-Val without any added proteins or peptides. The difference between baseline and the number of migrated cells with VEGF added equals maximal total migration.

Statistical Analysis. All results are expressed as mean ±SE. The paired Student's t test was used for comparison of eyes from the same animal. Groups were analyzed for differences by one-way ANOVA. Differences were considered statistically significant when P<0.05.

### Example 1

### PEDF Inhibits VEGF-Induced Retinal Vascular Permeability Qualitatively

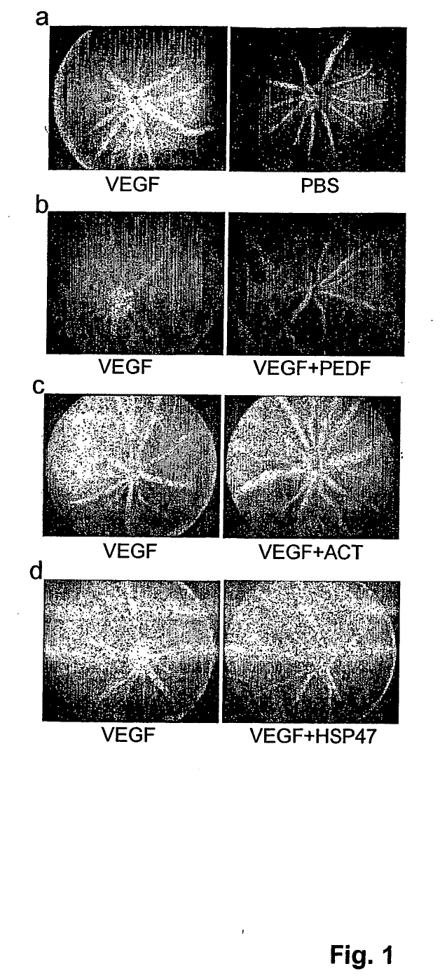

Fluorescein angiography, a clinical diagnostic technique, allows us to see photographically the effect of factors that modulate VEGF-induced permeability. Decreased fluorescence of one eye relative to the contralateral eye can be attributed to agents injected into the 2 eyes. Since VEGF promotes vascular permeability²⁸, there was, as expected, increased fluorescein leakage in the eye receiving VEGF₁₆₄ (the murine ortholog of human VEGF₁₆₅) when compared to the saline injected contralateral eye (FIG. 1a). The VEGF-induced vascular permeability was not observed when PEDF was co-injected with VEGF₁₆₄ (FIG. 1b).

To show that the anti-vasopermeability activity was specific to PEDF, we tested the effect of ACT and HSP47 in the same assay. ACT and HSP47 are from two subfamilies of the serpin superfamily²⁹, distinct from the subfamily to which PEDF belongs. Despite the high level of structural conservation among serpins^{30,31}, ACT and HSP47 had no effect on VEGF-induced fluorescein leakage in mouse retina (FIG. 1c, d). Thus, the inhibitory effect of PEDF on VEGF-induced vascular permeability is specific to PEDF.

### Example 2

### PEDF Inhibits VEGF-Induced Retinal Vascular Permeability Quantitatively

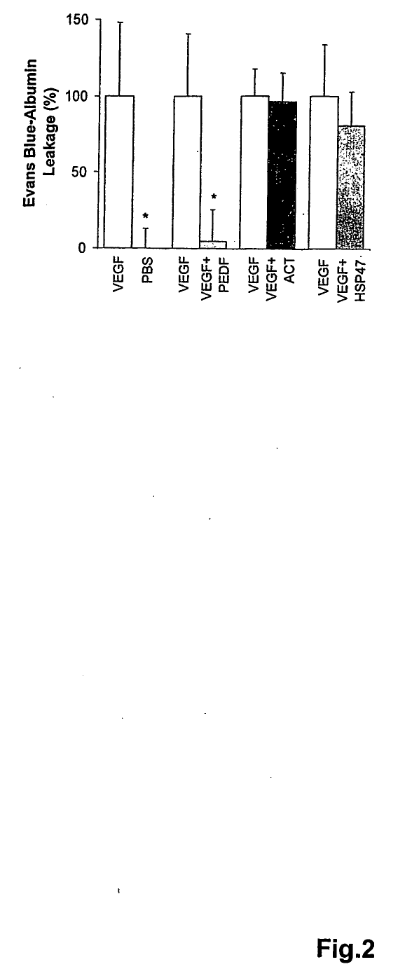

To quantify and confirm PEDF's ability to inhibit VEGF-induced vascular permeability, we used a modified Evans blue assay³². Mice, injected intravitreally as in the fluorescein angiography experiments, received intravascular Evans blue 24 hours later. PEDF nearly abolished (95.6±21.2%) the VEGF-induced permeability, whereas ACT and HSP47 had no discernible effect (inhibition of 3.4±18.2% and 19.4±22.3% respectively) (FIG. 2). These data corroborate quantitatively what we observed qualitatively by fluorescein angiography: PEDF inhibits VEGF-induced retinal vascular permeability.

### Example 3

### PEDFₚₑₚ Inhibits VEGF-Induced Vascular Permeability

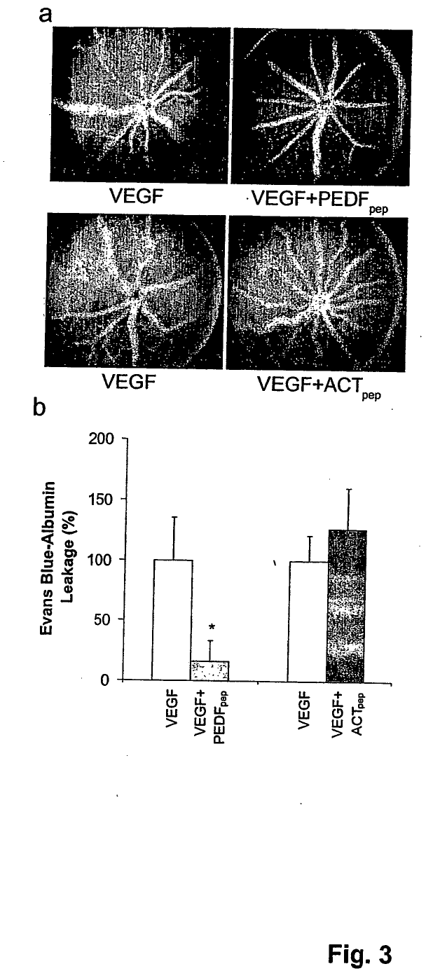

Because PEDF's neurotrophic/neuroprotective activity has been attributed to a 44-amino acid region^{24,25}, we asked whether this region also possesses the permeability modulating activity. PEDFₚₑₚ, which consists of amino acid residues 78-121 of human PEDF, was injected intravitreally in place of, and in equimolar amounts as full-length PEDF. The peptide effectively inhibited VEGF-induced vascular permeability in the fluorescein angiographic assay (FIG. 3a). A 46-amino acid peptide from the corresponding region of ACT (positions 73-118, designated ACTₚₑₚ) had no effect on VEGF-induced vascular permeability.

The Evans blue assay corroborated the fluorescein angiographic findings (FIG. 3b). PEDFₚₑₚ blocked 83.7±17.1% of VEGF-induced retinal vascular permeability to Evans blue-albumin. Similar to full-length ACT, ACTₚₑₚ, did not inhibit VEGF-induced vascular permeability (−26.4±34.3%). Full-length PEDF and PEDFₚₑₚ at equimolar concentrations were similarly potent. Analysis by one-way ANOVA showed no significant difference between their efficacies. The 44-amino acid region near the N-terminus of PEDF confers the inhibitory activity of PEDF on VEGF-induced vascular permeability.

### Example 4

### Four Amino Acid Residues within PEDF_{Pep} are Necessary for Inhibiting VEGF-Induced Vascular Permeability Activity

To identify the amino acid residues essential for the bioactivity, we compared the sequences and crystallographic structures of ACT, HSP47, and PEDF (FIGS. 4a, b), and selected 4 candidate amino acid residues within PEDF for evaluation as the key moieties. Previous work^{24,25} and our preliminary studies pointed to residues 78-121 of PEDF as the active site. From the crystal structure, the 44-amino acid region includes the complete secondary structural elements s6B, hB and hC, one turn of hD, and the connecting loops³¹. Both s6B and hB are buried in the interior of PEDF. The elements hC, hD, and the loop connecting them are largely exposed, forming an accessible surface. For this reason, we focused on residues 99-121, which contain hC, the connecting loop, and one turn of hD.

We reasoned that the key amino acids should be residues divergent between PEDF and the two serpins devoid of vascular permeability modulating activity (ACT and HSP47) (FIG. 4a). On this basis, 6 amino acids were identified. Two of these 6 amino acid residues were excluded. Arginine₉₉ was excluded because its change to alanine did not modify PEDF bioactivity (unpublished results). Proline₁₁₆ was excluded because of proline's role in maintaining the structure of the peptide backbone. The other 4 residues in PEDFₚₑₚ (glutamate₁₀₁, isoleucine₁₀₃, leucine₁₁₂, and serine₁₁₅) were modified to create CHIMERAₚₑₚ. Analogous to alanine scanning, glutamate₁₀₁ was replaced with alanine, the corresponding residue in HSP47. Isoleucine₁₀₃, leucine₁₁₂, and serine₁₁₅ were substituted with glutamate, the corresponding residues in ACT. At these 3 residues, ACT and HSP47 share similar electron rich side groups (glutamine or aspartate in HSP47).

In both the fluorescein angiographic assay (FIG. 4c) and the Evans blue assay (FIG. 4a), CHIMERAₚₑₚ failed to inhibit VEGF-induced vascular permeability. CHIMERAₚₑₚ inhibited the VEGF-induced Evans blue-albumin leakage by only 16.0±27.8%. In a one-way ANOVA test, CHIMERAₚₑₚ was significantly less effective than PEDF in the inhibition of VEGF-induced vascular permeability.

### Example 5

### The Same Region of PEDF Inhibits VEGF₁₆₄ Stimulated Endothelial Cell Migration

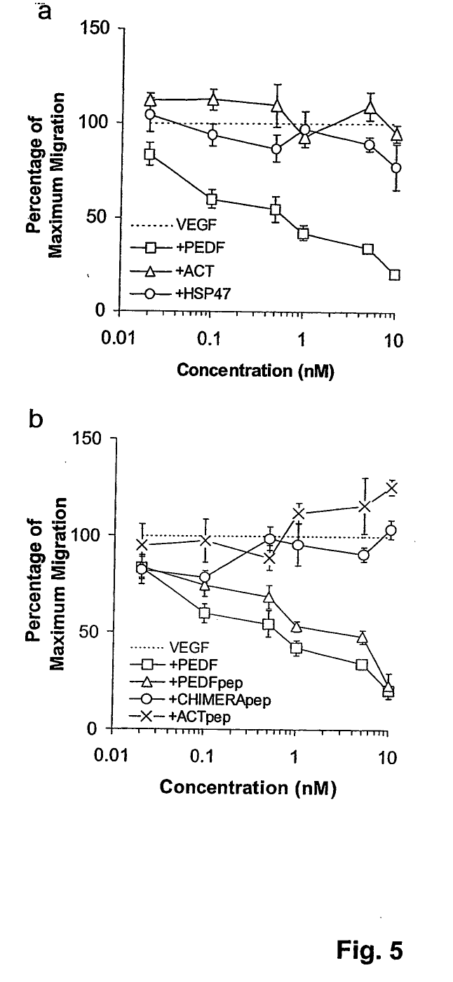

We used the microchemotaxis chamber assay with bovine retinal capillary endothelial cell (BRCEC) as a surrogate for angiogenic activity. PEDF inhibited VEGF-stimulated endothelial cell migration in a dose-dependent manner, with a half-maximal inhibitory concentration (IC₅₀) of 0.5 mM (FIG. 5a). ACT and HSP47 did not show effects on migratory activity at similar concentrations. Similar to PEDF, PEDFₚₑₚ effectively inhibited the VEGF-stimulated migration of BRCEC in a dose-dependent manner with a IC₅₀ of 3.0 nM. Neither ACTₚₑₚ nor CHIMERAₚₑₚ showed any effect in the same assay (FIG. 5b). Thus, endothelial cell migration depends on the same 4 amino acid residues.

All publications and patent applications disclosed herein are incorporated into this application by reference in their entirety, which can be used in the instant invention.

One skilled in the art will readily appreciate that the present invention is well adapted to carry out the objects and obtain the ends and advantages mentioned, as well as those inherent therein. It will be apparent to those skilled in the art that various modifications and variations can be made in practicing the present invention without departing from the spirit or scope of the invention. Changes therein and other uses will occur to those skilled in the art which are encompassed and equivalent within the spirit of the invention as defined by the scope of the claims.

### REFERENCES

1. Senger, D. R. et al. Tumor cells secrete a vascular permeability factor that promotes accumulation of ascites fluid. Science 219, 983-5 (1983).
2. Connolly, D. T. et al. Tumor vascular permeability factor stimulates endothelial cell growth and angiogenesis. J Clin Invest 84, 1470-8 (1989).
3. Keck, P. J. et al. Vascular permeability factor, an endothelial cell mitogen related to PDGF. Science 246, 1309-12 (1989).
4. Oosthuyse, B. et al. Deletion of the hypoxia-response element in the vascular endothelial growth factor promoter causes motor neuron degeneration. Nat Genet. 28, 131-8 (2001).
5. Sondell, M., Lundborg, G. & Kanje, M. Vascular endothelial growth factor has neurotrophic activity and stimulates axonal outgrowth, enhancing cell survival and Schwann cell proliferation in the peripheral nervous system. J Neurosci 19, 5731-40 (1999).
6. Wick, A. et al. Neuroprotection by hypoxic preconditioning requires sequential activation of vascular endothelial growth factor receptor and Akt. J Neurosci 22, 6401-7 (2002).
7. Waltenberger, J., Claesson-Welsh, L., Siegbahn, A., Shibuya, M. & Heldin, C. H. Different signal transduction properties of KDR and Flt1, two receptors for vascular endothelial growth factor. J Biol Chem 269, 26988-95 (1994).
8. Gille, H. et al. Analysis of biological effects and signaling properties of Flt-1 (VEGFR-1) and KDR (VEGFR-2). A reassessment using novel receptor-specific vascular endothelial growth factor mutants. J Biol Chem 276, 3222-30 (2001).
9. Bernatchez, P. N., Soker, S. & Sirois, M. G. Vascular endothelial growth factor effect on endothelial cell proliferation, migration, and platelet-activating factor synthesis is Flk-1-dependent. J Biol Chem 274, 31047-54 (1999).
10. Steele, F. R., Chader, G J., Johnson, L. V. & Tombran-Tink, J. Pigment epithelium-derived factor: neurotrophic activity and identification as a member of the serine protease inhibitor gene family. Proc Nat Acad Sci USA 90, 1526-30 (1993).
11. Becerra, S. P., Sagasti, A., Spinella, P. & Notario, V. Pigment epithelium-derived factor behaves like a noninhibitory serpin. Neurotrophic activity does not require the serpin reactive loop. J Biol Chem 270, 25992-9 (1995).
12. Dafform, T. R., Della, M. & Miller, A. D. The molecular interactions of heat shock protein 47 (Hsp47) and their implications for collagen biosynthesis. J Biol Chem 276, 49310-9 (2001).
13. Tombran-Tink, J. & Johnson, L. V. Neuronal differentiation of retinoblastoma cells induced by medium conditioned by human RPE cells. Invest Opthalmol V is Sci 30, 1700-7 (1989).
14. Tombran-Tink, J., Chader, G. G. & Johnson, L. V. PEDF: a pigment epithelium-derived factor with potent neuronal differentiative activity. Exp Eye Res 53, 411-4 (1991).
15. Becerra, S. P. et al. Overexpression of fetal human pigment epithelium-derived factor in Escherichia coli. A functionally active neurotrophic factor. J Biol Chem 268, 23148-56 (1993).
16. Seigel, G. M. et al. Differentiation of Y79 retinoblastoma cells with pigment epithelial-derived factor and interphotoreceptor matrix wash: effects on tumorigenicity. Growth Factors 10, 289-97 (1994).
17. Dawson, D. W. et al. Pigment epithelium-derived factor: a potent inhibitor of angiogenesis. Science 285, 245-8 (1999).
18. Stellmach, V., Crawford, S. E., Zhou, W. & Bouck, N. Prevention of ischemia-induced retinopathy by the natural ocular antiangiogenic agent pigment epithelium-derived factor. Proc Natl Acad Sci USA 98, 2593-7 (2001).
19. Duh, E. J. et al. Pigment epithelium-derived factor suppresses ischemia-induced retinal neovascularization and VEGF-induced migration and growth. Invest Opthalmol Vis Sci 43, 821-9 (2002).
20. Gao, G. et al. Difference in ischemic regulation of vascular endothelial growth factor and pigment epithelium-derived factor in brown norway and sprague dawley rats contributing to different susceptibilities to retinal neovascularization. Diabetes 51, 1218-25 (2002).
21. Gao, G. et al. Down-regulation of vascular endothelial growth factor and up-regulation of pigment epithelium-derived factor: a possible mechanism for the anti-angiogenic activity of plasminogen kringle 5. J Biol Chem 277, 9492-7 (2002).
22. Gao, G. et al. Unbalanced expression of VEGF and PEDF in ischemia-induced retinal neovascularization. FEBS Lett 489, 270-6 (2001).
23. Ohno-Matsui, K. et al. Novel mechanism for age-related macular degeneration: an equilibrium shift between the angiogenesis factors VEGF and PEDF. J Cell Physiol 189, 323-33 (2001).
24. Alberdi, E., Aymerich, M. S. & Becerra, S. P. Binding of pigment epithelium-derived factor (PEDF) to retinoblastoma cells and cerebellar granule neurons. Evidence for a PEDF receptor. J Biol Chem 274, 31605-12 (1999).
25. Bilak, M. M. et al. Identification of the neuroprotective molecular region of pigment epithelium-derived factor and its binding sites on motor neurons. J Neurosci 22, 9378-86 (2002).
26. Cao, W. et al. Pigment epithelium-derived factor protects cultured retinal neurons against hydrogen peroxide-induced cell death. J Neurosci Res 57, 789-800 (1999).
27. Taniwaki, T., Becerra, S. P., Chader, G. J. & Schwartz, J P. Pigment epithelium-derived factor is a survival factor for cerebellar granule cells in culture. J Neurochem 64, 2509-17 (1995).
28. Derevjanik, N. L. et al. Quantitative assessment of the integrity of the blood-retinal barrier in mice. Invest Opthalmol Vis Sci 43, 2462-7 (2002).
29. Gettins, P. G. Serpin structure, mechanism, and function. Chem Rev 102, 4751-804 (2002).
30. Becerra, S. P. Structure-function studies on PEDF. A noninhibitory serpin with neurotrophic activity. Adv Exp Med Biol 425, 223-37 (1997).
31. Simonovic, M., Gettins, P. G. & Volz, K. Crystal structure of human PEDF, a potent anti-angiogenic and neurite growth-promoting factor. Proc Natl Acad Sci USA 98, 11131-5 (2001).
32. Xu, Q., Qaum, T. & Adamis, A. P. Sensitive blood-retinal barrier breakdown quantitation using Evans blue. Invest Opthalmol V is Sci 42, 789-94 (2001).
33. Conn, G. et al. Purification of a glycoprotein vascular endothelial cell mitogen from a rat glioma-derived cell line. Proc Natl Acad Sci USA 87, 1323-7 (1990).
34. Gospodarowicz, D., Abraham, J A. & Schilling, J. Isolation and characterization of a vascular endothelial cell mitogen produced by pituitary-derived folliculo stellate cells. Proc Natl Acad Sci USA 86, 7311-5 (1989).
35. Leung, D. W., Cachianes, G., Kuang, W. J., Goeddel, D. V. & Ferrara, N. Vascular endothelial growth factor is a secreted angiogenic mitogen. Science 246, 1306-9 (1989).
36. Gettins, P. G., Simonovic, M. & Volz, K. Pigment epithelium-derived factor (PEDF), a serpin with potent anti-angiogenic and neurite outgrowth-promoting properties. Biol Chem 383, 1677-82 (2002).
37. Clermont, A. C., Cahill, M. T., Bursell, S. E., Bouck, N. & Aiello, L. P. Pigment epithelium-derived factor (PEDF) inhibits vascular endothelial growth factor (VEGF)-induced retinal permeability and blood flow in vivo. Invest Opthalmol V is Sci 42, S92 (2001).
38. Aiello, L. P. & Wong, J. S. Role of vascular endothelial growth factor in diabetic vascular complications. Kidney Int Suppl 77, S113-9 (2000).
39. Meyer, C., Notari, L. & Becerra, S. P. Mapping the type I collagen binding site on pigment epithelium-derived factor. Implications for its antiangiogenic activity. J Biol Chem (2002).
40. Roberts, D. L., Weix, D. J., Dahms, N. M. & Kim, J. J. Molecular basis of lysosomal enzyme recognition: three-dimensional structure of the cation-dependent mannose 6-phosphate receptor. Cell 93, 63948 (1998).
41. Tong, P. Y., Gregory, W. & Kornfeld, S. Ligand interactions of the cation-independent mannose 6-phosphate receptor. The stoichiometry of mannose 6-phosphate binding. J Biol Chem 264, 7962-9 (1989).
42. Tong, P. Y. & Kornfeld, S. Ligand interactions of the cation-dependent mannose 6-phosphate receptor. Comparison with the cation-independent mannose 6-phosphate receptor. J Biol Chem 264, 7970-5 (1989).
43. Stratikos, E., Alberdi, E., Gettins, P. G. & Becerra, S. P. Recombinant human pigment epithelium-derived factor (PEDF): characterization of PEDF overexpressed and secreted by eukaryotic cells. Protein Sci 5, 2575-82 (1996).
44. Qaum, T. et al. VEGF-initiated blood-retinal barrier breakdown in early diabetes. Invest Opthalmol V is Sci 42, 2408-13 (2001).
45. Gardner, T. W. Histamine, ZO-1 and increased blood-retinal barrier permeability in diabetic retinopathy. Trans Am Opthalmol Soc 93, 583-621 (1995).

We claim:

1. A method of treating a patient with a condition involving increased vascular permeability comprising administering to the patient a therapeutically effective amount of PEDF.

2. The method of claim 1 wherein PEDF is comprised of the amino acid sequence of SEQ ID NO.: 1.

3. The method of claim 1 wherein the condition is sepsis.

4. The method of claim 1 wherein the condition is acute respiratory distress syndrome.

5. The method of claim 1 wherein the condition is nephrotic syndrome.

6. The method of claim 1 wherein the condition is diabetic nephropathy.

7. The method of claim 1 wherein the condition is preproliferative diabetic retinopathy.

8. A method of treating a patient with a condition involving increased vascular permeability comprising administering to the patient a therapeutically effective amount of PEDF 44 AA peptide.

9. The method of claim 8 wherein PEDF 44 M peptide is comprised of the amino acid sequence of SEQ ID NO.: 2.

10. The method of claim 8 wherein the condition is sepsis.

11. The method of claim 8 wherein the condition is acute respiratory distress syndrome.

12. The method of claim 8 wherein the condition is nephrotic syndrome.

13. The method of claim 8 wherein the condition is diabetic nephropathy.

14. The method of claim 8 wherein the condition is preproliferative diabetic retinopathy.

15. A method of treating a patient with a condition involving increased vascular permeability comprising administering to the patient a therapeutically effective amount of a homolog of the PEDF 44 AA peptide wherein amino acid residues glutamate at the 101 amino acid position, isoleucine at the 103 amino acid position, leucine at the 112 and serine at the 115 amino acid position are unchanged.

16-21. (canceled)

23. A method of treating a patient with a condition involving increased vascular permeability comprising administering to the patient a therapeutically effective amount of an agent that activates the PEDF receptor.

24-29. (canceled)

30. A method of decreasing vascular permeability within a tissue, said method comprising providing exogenous PEDF to endothelial cells associated with said tissue under conditions sufficient for said PEDF to decrease vascular permeability within said tissue.

31. A method of decreasing vascular permeability within a tissue, said method comprising providing exogenous PEDF 44 AA peptide to endothelial cells associated with said tissue under conditions sufficient for said PEDF 44 AA peptide to decrease vascular permeability within said tissue.

32. A method of decreasing vascular permeability within a tissue, said method comprising providing an exogenous homolog of PEDF 44 AA peptide to endothelial cells associated with said tissue under conditions sufficient for said PEDF 44 AA peptide to decrease vascular permeability within said tissue.

33-36. (canceled)

37. A method of treating a patient with a condition involving increased angiogenesis comprising administering to the patient a therapeutically effective amount of PEDF 44 AA peptide.

38-40. (canceled)

41. A method of treating a patient with a condition involving increased angiogenesis comprising administering to the patient a therapeutically effective amount of a homolog of the PEDF 44 AA peptide wherein amino acid residues glutamate at the 101 amino acid position, isoleucine at the 103 amino acid position, leucine at the 112 and serine at the 115 amino acid position are unchanged.

42-45. (canceled)

45. A method of treating a patient with a condition involving increased angiogenesis comprising administering to the patient a therapeutically effective amount of an agent that activates the PEDF receptor.

46-48. (canceled)

49. A method of decreasing angiogenesis within a tissue, said method comprising providing exogenous PEDF 44 AA peptide to endothelial cells associated with said tissue under conditions sufficient for said PEDF 44 AA peptide to vascular permeability within said tissue.

50. A method of decreasing angiogenesis within a tissue, said method comprising providing an exogenous homolog of PEDF 44 AA peptide to endothelial cells associated with said tissue under conditions sufficient for said PEDF 44 AA peptide to vascular permeability within said tissue.

51-53. (canceled)

54. A method of treating a patient with a condition involving a neuropathy comprising administering to the patient a therapeutically effective amount of PEDF 44 M peptide.

55-57. (canceled)
